(19) United States
(12) Patent Application Publication
(10) Pub. No.: US 2007/0244441 A1
(43) Pub. Date: Oct. 18, 2007

(54) Intravenous needle assembly and method of use

(76) Inventor: Joshua L. Abbott, Broken Arrow, OK (US)

Correspondence Address: HEAD, JOHNSON & KACHIGIAN, 228 W 17TH PLACE, TULSA, OK, 74119, US

(21) Appl. No.: 11/403,330

(22) Filed: Apr. 13, 2006

(51) Int. Cl. A61M 5/32

(52) U.S. Cl. 604/174

(57) ABSTRACT

An intravenous needle assembly includes a compressible member for uses such as drawing blood and infusing intravenous fluids or medication. The intravenous needle assembly also includes an intravenous needle, a body to which the compressible member is attached, a fastener for maintaining a desired position of the needle during use, an attachment mechanism and a fluid conduit in fluid communication with the intravenous needle and attachment mechanism. In use, the needle is inserted into a patient's vein. The compressible member is compressed to position the needle as desired, and the fastener is employed to secure the needle assembly to the patient in a way that maintains compression on the compressible member. The needle assembly may be further secured to the patient by an adhesive disposed on the bottom surface of the compressible member. The attachment mechanism may be attached to a container for blood collection.

### REFERENCE TO PENDING APPLICATIONS

This application is not based upon any pending domestic or international patent applications.

### REFERENCE TO MICROFICHE APPENDIX

This application is not referenced in any microfiche appendix.

### FIELD OF THE INVENTION

The present invention generally relates to intravenous needles. More specifically, the present invention relates to an intravenous needle uniquely configured to maintain the needle at a desired position during use.

### BACKGROUND OF THE INVENTION

There are many uses for intravenous needles in the medical industry. Some of the more common uses of intravenous needles include administering medicine, drawing blood samples, and providing patients with intravenous fluids.

Intravenous needles are available in various configurations. So-called “butterfly” needles include wings or tabs that extend out perpendicular to the needle to help secure and steady the needle when the wings are grasped by the care provider. A small diameter hose having an attachment mechanism at one end is in fluid communication with the needle. The attachment mechanism may include a second needle for penetrating the seal on an IV fluid line or a container used to collect a blood sample. This configuration of needle assembly and attachment mechanism can be used to collect multiple blood samples by changing out the container attached to the attachment mechanism.

One of the shortcomings of prior art butterfly needles is the difficulty associated with maintaining the needle at a proper position in the patient's vein during use. Even a small amount of needle movement can place unwanted stress on the vein and cause the needle to rupture the vein or to detach from the vein. When using a butterfly needle to collect blood, the care provider must hold the needle in place with one hand while collecting blood samples with the other hand—a difficult procedure even for experienced care providers. Since the depth and location of veins below the skin vary from patient to patient, optimal positioning of the needle (particularly the angle at which the needle penetrates the skin and vein) will also vary. Unfortunately, intravenous needles available today do not effectively or efficiently maintain proper needle positioning. Instead, the position of the needle during use is dependent on the inconsistent ability of the care provider to hold the needle in place.

What is needed, therefore, is an intravenous needle capable of effectively maintaining proper position of the needle during use while enabling the care provider to use both hands to attend to tasks other than holding the needle in place.

### BRIEF SUMMARY OF THE INVENTION

The present invention achieves its objectives by providing an intravenous needle assembly that includes a needle with a central axis, a hollow interior extending along the central axis, and opposed first and second ends. A body having a top in opposed relation to a bottom is secured to the needle. A fluid conduit interconnects the second end of the needle with an attachment mechanism. A compressible member is attached to the needle assembly for being placed in a compressed state to obtain a desired position of the needle during use. A fastener is used to secure the needle assembly to the patient while the compressible member is in a compressed state so that compression of the compressible member is maintained during use.

A better understanding of the invention will be obtained from the following detailed description of the preferred embodiments taken in conjunction with the drawings and the attached claims.

### BRIEF DESCRIPTION OF THE DRAWINGS

Preferred embodiments of the invention will now be described in further detail. Other features, aspects, and advantages of the present invention will become better understood with regard to the following detailed description, appended claims, and accompanying drawings (which are not to scale) where:

FIG. 1 is a perspective view of an embodiment of the present invention;

FIG. 2 is a perspective view of an embodiment of the present invention while in use;

FIG. 3 is a partial sectional side view of one embodiment of the present invention in use on a patient with a deep vein; and

FIG. 4 is a partial sectional side view of the same embodiment of the invention in use on a patient with a shallower vein.

### DETAILED DESCRIPTION OF THE PREFERRED EMBODIMENTS

It is to be understood that the invention that is now to be described is not limited in its application to the details of the construction and arrangement of the parts illustrated in the accompanying drawings. The invention is capable of other embodiments and of being practiced or carried out in a variety of ways. The phraseology and terminology employed herein are for purposes of description and not limitation.

Turning now to FIG. 1, an intravenous needle assembly 20 according to the present invention includes an intravenous needle 22, a body 24, a fluid conduit 26 and an attachment mechanism 28. The intravenous needle 22 preferably has a generally cylindrical shape with a central axis 30, a first end 32 and a second end 34 in opposed relation to the first end 32. The intravenous needle 22 has a hollow interior extending along the central axis 30.

The body 24 is secured to the needle 22 and includes a top 36 in opposed relation to a bottom 38. The general shape or footprint of the body 24 can vary greatly. The embodiment shown in FIG. 1 has a first and second wing 40 and 42 extending perpendicular from the central axis 30 of the needle 22.

The needle 22 can be secured to the body 24 in various ways. In a preferred embodiment as shown in FIG. 1, the needle is friction fit to at least a portion of the body 24 where it is connected to the fluid conduit 26.

A compressible member 44 with a top surface 46 and an opposing bottom surface 48 is secured to the body 24 such that the top surface 46 of the compressible member 44 is secured to the bottom surface 38 of the body 24. The compressible member 44 is constructed from a resilient foam or other material capable of being compressed. One such foam material that may be used is visco-elastic memory foam. Other foam materials that may be used include foams made from silicon, various plastics, poly vinyl chloride (PVC) and polyethylene. Other compressible materials may be used as well.

To help maintain proper positioning of the needle during use, a fastener is provided to secure the needle assembly 20 to the patient while compressible member 44 is in a compressed state. In a preferred embodiment, the fastener includes one or more adhesive strips, or tape 47a, 47b secured to the top 36 of the body 24. After the needle 22 is inserted into the vein and member 44 is compressed to position the needle 22 at a desired depth and angle, the needle assembly 20 is secured in place by sticking adhesive strips 47a, 47b to the patient's skin. Preferably, the adhesive strips 47a, 47b include a removable film covering to protect the adhesive surfaces of the strips 47a, 47b prior to use.

If desired, an optional layer of adhesive 50 may be disposed on the bottom surface 48 of the compressible member 44. This layer of adhesive 50 can be used to further secure the needle assembly 20 to the skin of the patient.

The intravenous needle assembly 20 includes an attachment mechanism 28. While the attachment mechanism 28 can take any form commonly known in the art for attaching to containers or an intravenous line, the attachment mechanism 28 typically has a needle 52. The needle 52 has a first end 54 and a second end 56 with a cylindrical shape and hollow interior. The intravenous needle 22 and the attachment mechanism 28 are connected by a fluid conduit 26. The fluid conduit 26 has a hollow interior which is in fluid communication with the second end 34 of the intravenous needle 22 and the second end 56 of the attachment mechanism 28 needle 52.

FIG. 2 shows the intravenous needle assembly 20 in use on a patient to draw a blood sample. The first end 32 of the intravenous needle 22 is inserted into the arm A of a patient. The intravenous needle 22 extends through the skin S and into a vein located below the skin in the arm A of the patient. The needle assembly 20 is first secured in place to the skin S on the arm A by an adhesive 50 exposed on the bottom surface 48 of the compressible member 44. There are also two pieces of tape 47a, 47b to secure the needle assembly 20 to the arm A once the care provider has compressed compressible member 44 to position the needle 22 as desired. Thus, tape 47a, 47b secures the needle assembly 20 to the patient in a way that maintains compression on the compressible member 44 so as to maintain the desired position of the needle 22 during use without the care provider having to hold the needle assembly 20 in place. The blood flows through the needle 22, fluid conduit 26, attachment mechanism 28 and into a container C.

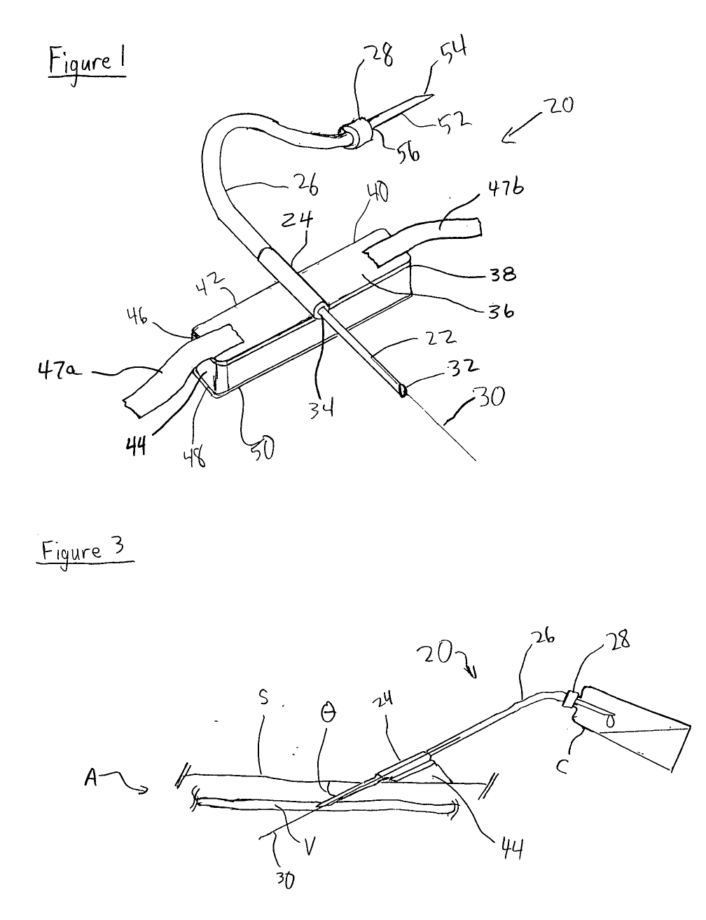

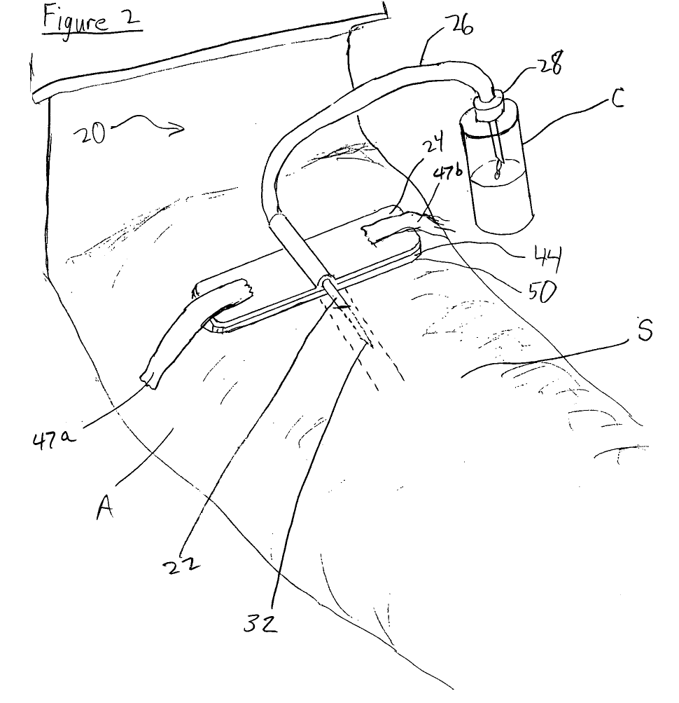

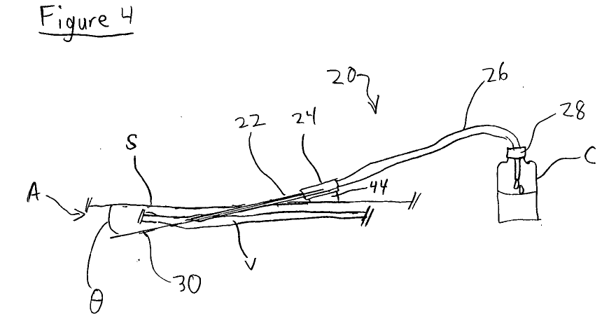

FIGS. 3 and 4 illustrate how the needle assembly 20 can be used or adapted to fit the particular vein depth during a procedure. FIG. 3 is a cross-sectional view showing the needle assembly 20 inserted into an arm A with a vein V that is relatively deep in relationship to the skin S covering the arm A. FIG. 4 provides a cross-sectional view of an arm A where the needle assembly 20 is used to obtain blood from a relatively shallow vein V below the skin surface S of the arm A. The angle of attack θ is the angle between the skin S of the patient's arm and the central axis 30 of the intravenous needle 22. As can be seen in comparing FIGS. 3 and 4, the angle of attack θ increases with the depth that the vein V lies beneath the skin S of the arm A.

In use, the intravenous needle 20 is inserted through the skin S of the patient's arm A and into a vein V at an angle of attack θ such that the needle 22 is long enough to penetrate the vein V. Once the intravenous needle 22 is inserted into the vein V, the compressible member 44 is compressed to position and support the intravenous needle 22 at the angle of attack θ. To maintain the needle 22 and body 24 at the angle of attack θ, the needle 22 and body 24 are held in place by adhesive strips 47a, 47b.

Once the needle 22 and body 24 are secured in place with adhesive strips 47a, 47b, the care provider may conveniently use both hands to attach a container C to the attachment mechanism 28 such that the vein V, intravenous needle 22, fluid conduit 26, attachment mechanism 28 and container C are all in fluid communication with one another. The higher pressure of the blood inside the vein V causes the blood to flow through the intravenous needle 22, fluid conduit 26, attachment mechanism 28 and into the container C where it collects. If necessary, the care provider can remove the first container C from the attachment mechanism 28 and replace it with a second container such that multiple samples of blood can be drawn without having to manually hold the needle assembly 20 in place. Once the desired amounts of blood has been drawn the care provider can then remove the intravenous needle 22 from the vein V and arm A. At the same time the body 24 and compressible member 44 are removed from the skin S of the arm A. It is then often necessary to apply pressure to the site where the needle 22 penetrated the skin S using a cotton ball or gauze bandage to control bleeding.

While this application has discussed the use of an intravenous needle assembly 20 in terms of drawing blood samples, the same apparatus could be used in a similar manner as a catheter or port for providing a patient with intravenous solutions or medication.

While the invention has been described with a certain degree of particularity, it is manifest that many changes may be made in the details of construction and the arrangement of components without departing from the spirit and scope of this disclosure. It is understood that the invention is not limited to the embodiments set forth herein for purposes of exemplification, but is to be limited only by the scope of the attached claims, including the full range of equivalency to which each element thereof is entitled.

What is claimed is:

1. An intravenous needle assembly comprising;
a needle having a central axis, a hollow interior extending along the central axis, a first end and a second end in opposed relation to the first end;
a body secured to said needle, said body having a top in opposed relation to a bottom;
an attachment mechanism;
a fluid conduit interconnecting the second end of said needle and said attachment mechanism;
a compressible member attached to said needle assembly for being placed in a compressed state to obtain a desired position of the needle during use, said compressible member having a top surface in opposed relation to a bottom surface, the top surface of said compressible member being secured to the bottom of said body, the bottom surface of said compressible member for being positioned adjacent the skin of a patient; and
a fastener for securing the needle assembly to the patient while said compressible member is in a compressed state, said needle assembly being secured by the fastener in a way that maintains compression on the compressible member.

2. The assembly of claim 1, wherein said fastener includes one or more pieces of tape.

3. The assembly of claim 1, wherein said body includes opposed left and right wings extending perpendicular from said central axis of said needle, each of said left and right wings having a top surface in opposed relation to a bottom surface with said compressible member being attached to the bottom surface of the wings, said fastener including a first piece of tape for securing the left wing to the patient and a second piece of tape for securing the right wing to the patient.

4. The assembly of claim 1, wherein said compressible member is fabricated from a material that slowly recovers its original shape after compression.

5. The assembly of claim 1, wherein said compressible member is fabricated from foam.

6. The assembly of claim 5, wherein said foam is a visco-elastic foam.

7. The assembly of claim 5, wherein said foam is poly vinyl chloride foam.

8. The assembly of claim 1 further comprising an adhesive disposed on the bottom surface of said compressible member for further securing the needle assembly to the patient.

9. The assembly of claim 1, wherein said attachment mechanism includes a needle having a cylindrical shape and a hollow interior, a first end and a second end, said first end in fluid communication with said fluid conduit.

10. An intravenous needle assembly comprising;
a needle having a central axis, a hollow interior extending along the central axis, a first end and a second end in opposed relation to the first end;
a body secured to said needle, said body having:
a top;
a bottom in opposed relation to the top;
a left wing extending perpendicular from said central axis of said needle and having opposed top and bottom surfaces; and
a right wing in opposed relation to said left wing and extending perpendicular from said central axis of said needle and having opposed top and bottom surfaces;
an attachment mechanism;
a fluid conduit interconnecting the second end of said needle and said attachment mechanism;
a compressible member attached to the bottom surfaces of said left and right wings for being placed in a compressed state to obtain a desired position of the needle during use, said compressible member having a top surface in opposed relation to a bottom surface, the top surface of said compressible member being secured to the bottom of said body, the bottom surface of said compressible member for being positioned adjacent the skin of a patient; and
a fastener for securing the needle assembly to the patient while said compressible member is in a compressed state, said fastener including:
a first piece of tape for securing the left wing to the patient; and
a second piece of tape for securing the right wing to the patient.

11. A process of using an intravenous needle assembly, the process comprising:
providing a needle assembly having:
a needle having a central axis, a hollow interior extending along the central axis, a first end and a second end in opposed relation to the first end;
a body secured to said needle, said body having a top in opposed relation to a bottom;
an attachment mechanism;
a fluid conduit interconnecting the second end of said needle and said attachment mechanism;
a compressible member attached to said needle assembly for being placed in a compressed state to obtain a desired position of the needle during use, said compressible member having a top surface in opposed relation to a bottom surface, the top surface of said compressible member being secured to the bottom of said body, the bottom surface of said compressible member for being positioned adjacent the skin of a patient; and
a fastener for securing the needle assembly to the patient while said compressible member is in a compressed state;
positioning the bottom surface of said compressible member against the skin of a patient;
inserting said first end of said needle through the skin a patient and into a vein;
compressing said compressible member to obtain a desired position of the needle for use; and
securing the needle assembly to the patient with the fastener in a way that maintains compression on the compressible member.

12. The process of claim 11, further comprising:
providing a container;
securing said attachment mechanism to said container such that said container is in fluid communication with said attachment mechanism, said fluid conduit, said needle and said vein; and
collecting blood in said container.

13. The process of claim 11, further comprising providing an adhesive disposed on the bottom surface of the compressible member to further secure the needle assembly to the patient.
